(19) United States
(12) Patent Application Publication
(10) Pub. No.: US 2016/0184372 A1
(43) Pub. Date: Jun. 30, 2016

(54) COMPOSITION COMPRISING LACTIC ACID BACTERIA FOR USE IN THE PREVENTIVE AND/OR CURATIVE TREATMENT OF BACTERIAL VAGINOSIS

(71) Applicant: PROBIOTICAL S.P.A., Novara No (IT)

(72) Inventor: Giovanni MOGNA, NOVARA (IT)

(21) Appl. No.: 14/891,321

(22) PCT Filed: May 14, 2014
(86) PCT No.: PCT/IB2014/000739

§ 371 Date: Nov. 13, 2015

(30) Foreign Application Priority Data
May 14, 2013 (IT) MI2013A000794

(51) Int. Cl. A61K 35/747; A61K 47/26; A61K 9/00; A61K 47/36

(52) U.S. Cl. CPC A61K 35/747; A61K 47/36; A61K 47/26; A61K 9/0034; A61K 2035/115

(57) ABSTRACT

A composition comprising lactic bacteria for topical vaginal use is described. The composition is effectively applicable in the preventive and/or curative treatment of vaginal infections caused by vaginal pathogens, with particular reference to Gardnerella vaginalis and/or coliform bacteria, responsible for bacterial vaginosis, even recurrent or relapsing.

The present invention relates to a composition comprising lactic bacteria for topical vaginal use. The composition of the present invention is effectively applicable in the preventive and/or curative treatment of vaginal infections caused by vaginal pathogens, with particular reference to Gardnerella vaginalis and/or coliform bacteria, responsible for bacterial vaginosis, even recurrent or relapsing.

It is known that the vaginal microbiota consists of a pool of endogenous microorganisms with various size and nature, which physiologically live as commensal colonizing the eco-environment, and which thus, by definition, form the ecosystem thereof. Such an ecosystem is represented, on the whole, by the epithelial cells lining the vagina and uterus, by the glandular ones secreting in the lumen of the organ and by the complex bacterial flora, which symbiotically interacts for maintaining a proper physiological status. The equilibrium condition thus obtained supports a dynamics which alternates with physiopathological phenomena, wherein the environment influences the microbiota, and the microorganisms, in turn, through a mainly ascending propagation, influence the reactivity of the cell-mediated immune defence. The vaginal microbial population is mixed and comprises about 50 bacterial species, both aerobic and anaerobic. Usually the 95% of the microbial component is represented by lactobacilli, which form part of the “Doderlein complex”, whereas the remaining 5% comprises commensal bacteria and opportunistic pathogens, among which Streptococcus epidermidis, Diphtheroids, Peptostreptococci, Bacteroides spp., Escherichia coli, Gardnerella vaginalis and Candida spp.

Therefore, the strains belonging to the genus Lactobacillus represent the prevalent component, as shown by both standard analytical and molecular techniques: the concentration of such microorganisms is of about 1×10⁹ CFU/ml, a value about 100-fold greater than the concentration of the same bacterial group per gram of intestinal content. The most represented species of lactobacilli are L. acidophilus, L. fermentum, L. plantarum, L. brevis, L. jensenii, L. casei, L. cellobiosus, L. leichmanii, L. delbrueckii and L. salivarius.

Several studies concerning the vaginal microbiota indicate that lactobacilli, usually predominantly present, play a key role in preventing colonization by unwanted microorganisms, among which those responsible for bacterial vaginosis, fungal infections, sexually transmitted diseases and urinary tract infections (UTI).

The vaginal wall cells have a main role in maintaining such an equilibrium, in particular the upper and middle layers of epithelium, the proliferation and maturation of which is hormone-related. The estrogen levels, in fact, affect the glycogen content of the cells of the vaginal epithelium and the metabolism of the latter mediates the synthesis of organic acids, reducing the pH of the lumen of the organ up to values comprised between 3.5 and 4.5. Due to this mechanism, the invasion of exogenous pathogens or the replication of harmless microorganisms, usually existing at vaginal level at low amounts, is hindered. In prepubescent subjects the glycogen content of the vaginal epithelium is low and the pH is about 7.

However, it is quite frequent that, due to different exogenous and endogenous factors, such as antibiotic intake, stress conditions, hormonal modulations related to pregnancy, menstrual cycle or the intake of high estrogen concentrations, an unbalance of this complex and equilibrated ecosystem occurs.

Therefore, there is a need for effectively intervene in order to re-equilibrate and balance the vaginal ecosystem for avoiding that the alteration of the microbiota equilibrium leads to a prevalence of opportunistic microorganisms which, no longer hindered by the action of the “Doderlein complex”, can cause vaginitis or vaginosis forms caused by opportunistic germs, among which Gardnerella and/or coliforms.

In postmenopausal women, in which the drop of estrogen levels leads to a reduction of the vaginal glycogen concentration, the vaginal pH increases and the anaerobic bacteria become dominant so that a significant proliferation of Enterobacteriaceae takes place.

Bacterial vaginosis is the most common vaginal infection among women during reproductive age and it is also frequent among pregnant women. Bacterial vaginosis represents more than 60% of the overall vaginal infections. The National Center for Health Statistics—National Health and Nutrition Survey (NHANES) in a survey carried out on more than 12.000 women in USA on 2001-2004 showed that the prevalence of bacterial vaginosis was 29.2%, but only 15.7% was symptomatic. A good percentage of vaginosis is thus asymptomatic, namely does not lead to symptoms typical of vaginal inflammations such as itching, burning and pain during sexual activity. Many of them, inter alia, do not either cause irritations, redness or swelling but, in any case, they cause an increase of vaginal discharges, with white or grayish secretions, characterized by a bad smell. Due to the fact that vaginal discharges substantially change depending on the menstrual cycle stage, the woman is often unable to understand that there is an ongoing infection and vaginosis is not diagnosed. The non-treatment of a vaginosis can lead to the onset of even severe vaginitis and infections, mainly in particularly sensitive subjects. Furthermore, it can contribute in transmitting infections through the sexual activity.

Therefore, there is a need to effectively intervene for preventing and/or curing the onset of vaginosis and, consequently, of vaginitis and bacterial infections.

The real cause of bacterial vaginosis is still presently an object of study and investigation. The main agent responsible for most of the cases is the Gram-variable bacterium Gardnerella vaginalis (80-85%) the only known species of genus Gardnerella, and the Gram-negative bacterium E. coli (10-15%). Additionally, the pathogenic bacterium E. coli is also responsible for aerobic vaginosis, which causes the risk of preterm births.

Nevertheless, a bacterial vaginosis onset is related to the simultaneous interaction with other risk factors. Among the most significant risk factors, for example, an improper personal hygiene (for example an undue use of vaginal douches), the use of antibiotics or intrauterine mechanical contraceptives such as the coil, previous pregnancies and some genetic predisposition have to be cited.

Women with bacterial vaginosis have abnormal vaginal discharges, with an unpleasant smell. The possible discharges are usually of white or grey color and can be quite fluid. Furthermore, affected women experience burning during urination and/or external genital itching. However, as already explained, a fair part of affected women do not have any symptom.

In most of the cases, bacterial vaginosis does not cause any severe problem. Nevertheless, some quite severe potential complications have to be taken into account such as, for example, the increased risk of HIV infection, in the event of exposure to the virus, or the risk of infection by etiological agents of other sexually transmitted diseases, among which the genital virus Herpes simplex, Chlamydia and gonorrhea.

Bacteria causing vaginosis can also infect the uterus and Fallopian tubes. Such a kind of infection is named pelvic inflammatory disease and can lead to infertility or impair the tubes, increasing the risk of ectopic pregnancies and infertility.

It is known that bacterial vaginosis can be treated with molecules with antibiotic activity, among which metronidazole or clindamycin are very common. Both can be used even during pregnancy, but the recommended dosage is different. Although these actions are generally considered effective in the treatment of acute infections, they are often unable to provide a significant protection against possible relapses, which may occur at various times after the end of the therapy. Furthermore, the antibiotic molecules presently available still represent an approach based on organic synthetic drugs and, as such, clearly extraneous to the physiological habitat of the vagina.

Thus, there is a need to effectively intervene for preventing and/or curing the onset of vaginosis and, consequently, of vaginitis and bacterial infections avoiding the use of synthetic molecules but, on the contrary, by adopting a treatment compatible with the physiological habitat of the vagina.

Moreover, there is a need to have a natural and effective barrier effect, in the case of both an acute episode and possible relapses, which continues over the time in order to ensure a long-term protection and assure a long-lasting effective preventive and/or curative treatment.

The Applicant, following to an extended and intense research activity, gave a response to the above-cited needs. In fact, the Applicant, after testing and studying many lactic bacteria, succeeded to select only some of them based on their specific activity in colonizing the vaginal mucosa and producing bacteriostatic and bactericidal molecules. The selected strains are effective against Gardnerella vaginalis and are anti-E. coli also performing at the same time an anti-inflammatory activity since they are strains producing interleukin 10 (IL-10) and interleukin 4 (IL-4). The selected strains having these specific proprieties (anti-E. coli and anti-inflammatory) are effective against bacterial vaginosis, vaginitis and related infections.

It is an object of the present invention a composition for use in the preventive and/or curative treatment of bacterial vaginosis, preferably of recurrent or relapsing vaginosis, having the characteristics as described in the appended claim.

Preferred embodiments of the present invention are contemplated in the following detailed description.

In the context of the present invention by composition is meant a pharmaceutical composition, or a supplement product or a medical device or a food composition.

It is an object of the present invention a strain of bacteria belonging to the species Lactobacillus fermentum deposited by Probiotical S.p.A. Company on 3 Jan. 2013 at the DSMZ center and having deposit number DSM 26955 (LF15) and a strain of bacteria belonging to the species Lactobacillus fermentum deposited by Probiotical S.p.A. Company on 3 Jan. 2013 at the DSMZ center and having deposit number DSM 26956 (LF16), for use in the preventive and/or curative treatment of bacterial vaginosis, vaginitis and vaginal infections related with coliform pathogenic bacteria and/or Gardnerella.

It is an object of the present invention a composition comprising a mixture of bacteria, which comprises or, alternatively, consists of strains of bacteria having both a specific antibacterial activity against Gardnerella vaginalis and E. coli and an anti-inflammatory activity with stimulation of the Interleukin IL-4 and Interleukin IL-10 production.

It is an object of the present invention a composition wherein said mixture comprises or, alternatively, consists of at least a strain of bacteria having a specific antibacterial activity against Gardnerella vaginalis combined with at least a strain having an antibacterial E. coli activity and an anti-inflammatory activity with stimulation of the Interleukin IL-4 and Interleukin IL-10 production.

It is an object of the present invention a composition comprising or, alternatively, consisting of a mixture of bacteria which comprises or, alternatively, consists of at least a strain of bacteria selected from the group comprising the strain Lactobacillus plantarum LMG P-21021-LP01 and the strain Lactobacillus plantarum LMG P-21020-LP02, combined with at least a strain of bacteria selected from the group comprising the strain of bacteria Lactobacillus fermentum DSM 26955 (LF15) and the strain of bacteria Lactobacillus fermentum DSM 26956 (LF16), for use in the preventive and/or curative treatment of bacterial vaginosis, vaginitis and vaginal infections related with coliform pathogens and/or Gardnerella; preferably for the treatment of recurrent or relapsing vaginosis.

The strain Lactobacillus plantarum LMG P-21021-LP01 was deposited by Mofin Srl Company on 16 Oct. 2001 at the BCCM LMG center.

The strain Lactobacillus plantarum LMG P-21020-LP02 was deposited by Mofin Srl Company on 16 Oct. 2001 at the BCCM LMG center.

In a preferred embodiment, the composition of the present invention comprising or, alternatively, consisting of a mixture of bacteria which comprises or, alternatively, consists of the strain of bacteria Lactobacillus plantarum LMG P-21021-LP01, combined with at least a strain of bacteria selected from the group comprising the strain of bacteria Lactobacillus fermentum DSM 26955 (LF15) and the strain of bacteria Lactobacillus fermentum DSM 26956 (LF16), for use in the preventive and/or curative treatment of bacterial vaginosis, vaginitis and vaginal infections related with coliform pathogens and/or Gardnerella; preferably for the treatment of recurrent or relapsing vaginosis.

Preferably, said composition comprising or, alternatively, consisting of a mixture of bacteria which comprises or, alternatively, consists of the strain of bacteria Lactobacillus plantarum LMG P-21021-LP01 and the strain of bacteria Lactobacillus fermentum DSM 26955 (LF15).

In another preferred embodiment, the composition of the present invention comprising or, alternatively, consisting of a mixture of bacteria which comprises or, alternatively, consists of the strain of bacteria Lactobacillus plantarum LMG P-21020-LP02, combined with at least a strain of bacteria selected from the group comprising the strain of bacteria Lactobacillus fermentum DSM 26955 (LF15) and the strain of bacteria Lactobacillus fermentum DSM 26956 (LF16), for use in the preventive and/or curative treatment of bacterial vaginosis, vaginitis and vaginal infections related with coliform pathogens and/or Gardnerella; preferably for the treatment of recurrent or relapsing vaginosis.

Preferably, said composition comprising or, alternatively, consisting of a mixture of bacteria which comprises or, alternatively, consists of the strain of bacteria Lactobacillus plantarum LMG P-21020-LP02 and the strain of bacteria Lactobacillus fermentum DSM 26956 (LF16).

In another embodiment, the composition of the present invention further comprises a strain of bacteria selected from the group comprising or, alternatively, consisting of a strain Lactobacillus fermentum DSM 18296 (LF07) deposited on 24 May 2006 by Probiotical S.p.A Company.

The composition of the present invention further comprises at least a gelling substance, preferably having a marked muco-adhesive property, such as a natural gum or a plant gum and/or a plant gelatin. The plant gum and/or the plant gelatin is preferably selected from the group comprising a tannate or a gelatin tannate, an alginate, a xyloglucan or xylogel, a guar gum, a tara gum, an acacia, carob, oat, bamboo fiber, citrus fruit fibers and glucomannans. Preferably said gelling substance is selected from galactomannans of natural origin. In a preferred embodiment, the gelling substance is selected from guar gums.

Additionally, the composition of the present invention further comprises at least a fiber with prebiotic activity selected from the group comprising inulin, fructo-oligosaccharides (FOS), galacto- and trans-galacto-oligosaccharides (GOS and TOS), gluco-oligosaccharides (GOSa), xylo-oligosaccharides, (XOS), chitosan-oligosaccharides (COS), soy-oligosaccharides (SOS), isomalto-oligosaccharides (IMOS), resistant starch, pectins, psyllium, arabino-galactans, gluco-mannans and galacto-mannans. In an embodiment, said fiber with prebiotic activity is selected from fructo-oligosaccharides (FOS), arabinogalactans or mixtures thereof. An embodiment of the present invention is represented by a composition, which comprises or, alternatively, consists of a strain of bacteria Lactobacillus fermentum DSM 26955 (LF15), a strain of bacteria Lactobacillus plantarum LMG P-21021 (LP01) and/or a guar gum and/or a prebiotic fiber selected from arabinogalactan and fructo-oligosaccharide (FOS) or mixtures thereof.

Another embodiment of the present invention is represented by a composition, which comprises or, alternatively, consists of a strain of bacteria Lactobacillus fermentum DSM 26956 (LF16), a strain of bacteria Lactobacillus plantarum LMG P-21020 (LP02) and/or a guar gum and/or a prebiotic fiber selected from arabinogalactan and fructo-oligosaccharide (FOS) or mixtures thereof.

The composition of the present invention may be in solid form, for topical vaginal use, as a tablet, a capsule, an ovule, a lozenge or granules or powder; or in liquid form, for topical vaginal use, as a solution, a liquid vaginal douche, a suspension or a gel.

The composition of the present invention contains excipients and technological additives, known to the skilled in the field, suitable for preparing a solid form for internal use, which is able to disintegrate and release the strains of bacteria after the hydration of said solid form inside the vaginal cavity.

In addition, the composition of the present invention is prepared by techniques and equipment known to the skilled in the field which is able to suitably adopt the compressibility, temperature and concentration parameters of the various excipients being used in order to avoid that the bacterial load contained in a solid form, for example a tablet or an ovule for internal use, is dramatically reduced over the time and/or that the bacterial cell suffers from the preparation process to such an extent to compromise its health condition and viability.

The composition of the present invention in the form of tablet or ovule has an optimum survival of the two lactobacilli both during the compression step and during the commercial life of the product at a storage temperature not greater than 25° C. The composition of the present invention in solid form for topical vaginal use has a shelf-life of 2 years at room temperature ensuring a minimum bacterial load of at least 400×10⁶ viable cells, for each strain of bacterium therein contained, per tablet or ovule.

The presence of the gelling agent guar gum, together with the strains of bacteria of the present invention L. fermentum LF15 and L. plantarum LP01 or L. fermentum LF16 and L. plantarum LP02, is able to rapidly establish an effective barrier effect against potential vaginal pathogens, with particular reference to Gardnerella vaginalis and/or coliform bacteria, well known causes of vaginosis, even relapsing. The efficacy is against bacterial vaginosis, mainly when is caused by Gardnerella vaginalis and/or coliform bacteria.

The selected strains L. fermentum LF15 and L. fermentum LF16 are effective against Gardnerella vaginalis, whereas the selected strains L. plantarum LP01 and L. plantarum LP02 are active against E. coli and exert at the same time an anti-inflammatory activity since they are strains producing interleukin 10 (IL-10) and interleukin 4 (IL-4).

The use of the muco-adhesive gelling agent, selected from those described above, such as for example guar gum (natural galactomannan), is able to rapidly establish, through the formation of a hydrogel directly in the vagina and after very few minutes from disaggregation of the tablet or ovule, a mechanical barrier effect against potential vaginal pathogens, with particular reference to Gardnerella vaginalis and coliform bacteria, among which Escherichia coli has a particular importance.

As soon as the muco-adhesive gelling agent, for example guar gum, hydrates in the vaginal environment, this mediates the formation of a gel having an optimum viscosity which, by evenly distributing over the mucosa, creates a physical-mechanical barrier action against the adhesion of the above-cited commensal or pathogenic bacteria.

The main component of guar gum is a galactomannan, a trisaccharide consisting of mannose and galactose units, specifically polymerized to form α-D-mannopyranosil chains linked by a β-D-(1-4) glycosidic bond and with molecular weight around 200-300 kDa, to form a straight 1-4 chain with short side 1-6 branches of galactose.

The composition of the present invention, in addition to the gelling agent for example the guar gum, contains the two specific microorganisms Lactobacillus fermentum LF15 and Lactobacillus plantarum LP01 or Lactobacillus fermentum LF16 and Lactobacillus plantarum LP02, at a concentration comprised from 1×10⁷ to 1×10¹⁰, preferably from 1×10⁸ to 1×10⁹ CFU/g (and in any case not lesser than 400×10⁶ of viable cells per strain, per tablet or ovule), which rapidly integrate in the vaginal microbiota restoring the “Doderlein” complex and, consequently, enhancing the barrier effect.

The composition of the present invention contains the two specific microorganisms Lactobacillus fermentum LF15 and Lactobacillus plantarum LP01 or Lactobacillus fermentum LF16 and Lactobacillus plantarum LP02, in a ratio comprised from 3:1 to 1:3, preferably from 2:1 to 2:1, even more preferably 1:1; the gelling substance in an amount comprised from 1 to 50% by weight, preferably from 5 to 35%, even more preferably from 10 to 20%; the prebiotic fiber in an amount comprised from 1 to 50% by weight, preferably from 5 to 35%, even more preferably from 10 to 20%.

In fact, the bacteria of the present invention are able to establish a long-term effective barrier effect due to the colonization of the vaginal mucosa, which leads to a self-regulated production of organic acids, mainly lactic and acetic. These bacteria enhance the barrier effect against commensal or pathogenic bacteria responsible for vaginosis through the synthesis and release, by the two lactobacilli, of molecules with specific activity, mainly bacteriolysins and bacteriocins, in addition to the organic acids (lactic and acetic).

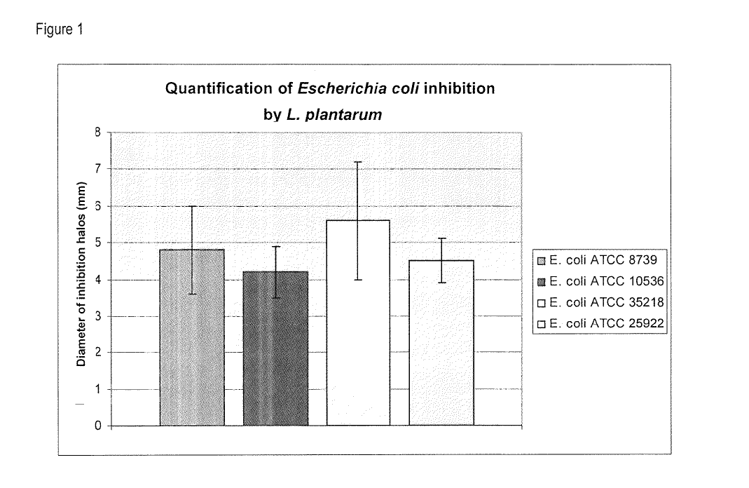

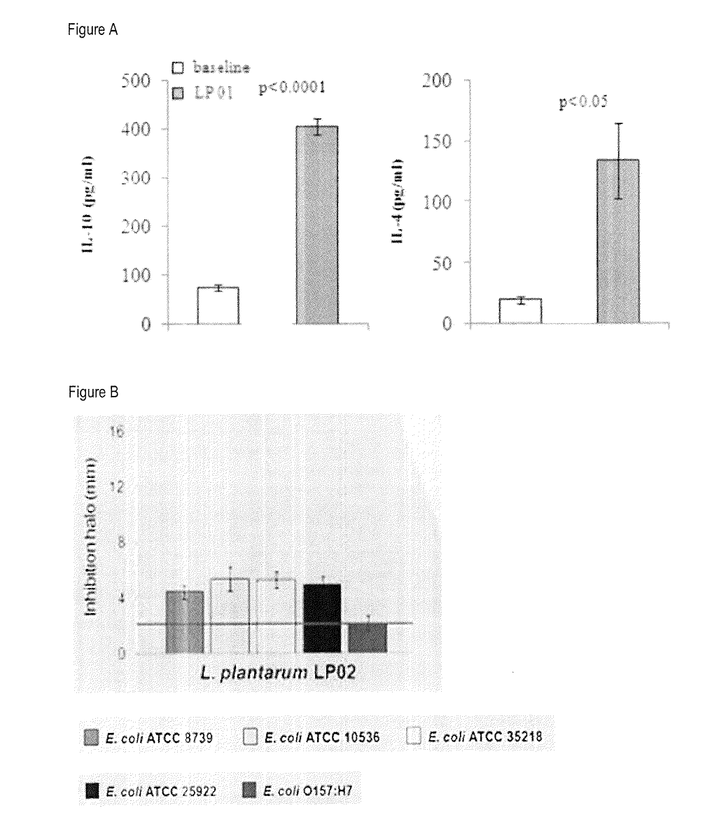

The antagonistic activity of the strain L. plantarum LP01 and the strain L. plantarum LP02 was tested in vitro against 4 strains of Escherichia coli (ATCC 8739, ATCC 10536, ATCC 35218 and ATCC 25922). Only the results relative to the strain L. plantarum LP01 (FIG. 1) are presented, since both led to similar results.

The results are disclosed in FIG. 1. The results are expressed as diameter of inhibition halo (mm) mediated by the lactobacillus on each strain of Escherichia coli. The significance threshold of the results was taken equal to 2 mm. An inhibition halo is an area of the dish wherein E. coli was unable to grow due to the barrier effect exerted by L. plantarum LP01. The results are disclosed as means±standard deviation (SD) of 3 independent experiments.

As it can be noted from histograms (FIG. 1), the strain L. plantarum LP01 is able to significantly hinder all of the 4 strains of Escherichia coli being tested, with inhibition diameters greater than 4 mm.

On the other hand, the strains of bacteria L. fermentum LF15 and L. fermentum LF16 are able to significantly hinder the rod-shaped, Gram-variable bacterium Gardnerella vaginalis. In particular, the antagonistic activity of said lactobacilli was tested against a strain of Gardnerella, by quantification of the optical density detected at the wavelength of 600 nm (OD₆₀₀) after 24 and 48 hours of microaerophilic growth. Gardnerella was grown in a liquid TH medium, whereas each lactobacillus tested in the experiment was cultured in a liquid MRS medium. At the end of the growing, the neutralized supernatants from the cultures of each lactobacillus were added at different percentages to fresh MRS inoculated with Gardnerella vaginalis 2%. The growing of Gardnerella alone in fresh MRS (positive control) and with neutralized supernatant amounts comprised between 0.5% and 4%, after 24 and 48 hours. Only the results relative to the strain L. fermentum LF15 (Table 1 at 24 hours and Table 2 at 48 hours) are shown, since similar results were also obtained for the strain L. fermentum LF16.

TABLE 1

Fresh MRS amount

| 24 hours | 0.5 | 1 | 2 | 4 |
|---|---|---|---|---|
| Gardnerella vaginalis | 1.251 | 1.534 | 1.811 | 1.807 |

| | Supernatant amount | | | |
|---|---|---|---|---|
| Neutralized supernatant | 0.5 | 1 | 2 | 4 |
| L. crispatus DSM 24439 (ID 1626) | 0.505 | 0.511 | 0.673 | 1.035 |
| L. crispatus DSM 24438 (ID 1605) | 0.531 | 0.484 | 0.462 | 0.511 |
| L. paracasei DSM 21718 LPC08 | 0.653 | 1.130 | 1.538 | 1.724 |
| (ID 1696) | | | | |
| L. paracasei DSM 24440 (ID 1608) | 0.689 | 1.214 | 1.476 | 1.774 |
| L. fermentum DSM 18289 (ID 1456) | 0.727 | 0.541 | 0.591 | 0.745 |
| L. fermentum DSM 18296 (ID 1459) | 0.677 | 0.530 | 0.589 | 0.665 |
| L. fermentum DSM 18297 (ID 1460) | 0.681 | 0.583 | 0.717 | 0.710 |
| L. fermentum DSM 26955 (LF15) | 0.255 | 0.339 | 0.302 | 0.321 |
| L. fermentum DSM 26956 (LF16) | 0.483 | 0.425 | 0.486 | 0.416 |

TABLE 2

Fresh MRS amount

| 48 hours | 0.5 | 1 | 2 | 4 |
|---|---|---|---|---|
| Gardnerella vaginalis | 0.874 | 1.255 | 1.874 | 1.837 |

| | Supernatant amount | | | |
|---|---|---|---|---|
| Neutralized supernatant | 0.5 | 1 | 2 | 4 |
| L. crispatus DSM 24439 (ID 1626) | 0.437 | 0.465 | 0.508 | 0.509 |
| L. crispatus DSM 24438 (ID 1605) | 0.367 | 0.369 | 0.362 | 0.380 |
| L. paracasei DSM 21718 LPC08 | 0.489 | 0.625 | 1.082 | 1.734 |
| (ID 1696) | | | | |
| L. paracasei DSM 24440 (ID 1608) | 0.522 | 0.591 | 0.879 | 1.748 |
| L. fermentum DSM 18289 (ID 1456) | 0.617 | 0.463 | 0.435 | 0.446 |
| L. fermentum DSM 18296 (ID 1459) | 0.489 | 0.474 | 0.379 | 0.370 |
| L. fermentum DSM 18297 (ID 1460) | 0.510 | 0.532 | 0.472 | 0.438 |
| L. fermentum DSM 26955 (LF15) | 0.255 | 0.317 | 0.233 | 0.212 |
| L. fermentum DSM 26956 (LF16) | 0.366 | 0.348 | 0.312 | 0.287 |

All the strains of bacterium listed in Table 1 and Table 2 were deposited by the Applicant and are available to the public in accordance with the requirements established by the Budapest Treaty. As expected, the growth of G. vaginalis alone in fresh MRS medium is directly proportional to the percentage of medium itself made available to the bacterium. When increasing the percentage of neutralized supernatant, namely brought to neutral pH in order to eliminate the non-specific inhibitory effects mediated by the organic acids produced by lactobacilli, the growth of G. vaginalis resulted decreased mainly in the case of L. fermentum LF15, capable to mediate a totally specific inhibitory action. On the other hand, with L. paracasei LPC08 and L. paracasei ID 1608 the growing of G. vaginalis had values substantially similar to those obtained in fresh MRS, suggesting that these two lactobacilli exert no inhibitory action. The remaining five strains mediate an antagonistic action, mainly L. crispatus ID 1605 and L. fermentum ID 1459, although of a lesser extent than L. fermentum LF15. The same applies for the strain L. fermentum LF16.

In addition to the above-described specific inhibitory mechanisms, both lactobacilli are able to produce high amounts of lactic and acetic acids due to their facultative heterofermentative metabolism, contributing to rapidly and considerably lower the intravaginal pH.

Furthermore, the presence of the two fibers, such as for example arabinogalactan and short chain fructo-oligosaccharides (FOSsc) provides a selective nourishment for the microorganisms of the “Doderlein complex” and lactobacilli existing in the formulation, thereby promoting the ability of colonizing the vaginal ecosystem and the subsequent development.

The competitive advantage deriving from the capability of fermenting these particular carbon sources results in a fast increase of the L. fermentum LF15, L. plantarum LP01, L. fermentum LF16 and L. plantarum LP02 and other autochthonous lactobacilli populations, with subsequent synthesis of remarkable amounts of peptides with antimicrobial action and organic acids able to lowering the vaginal pH to values incompatible with most of the pathogenic species.

Advantageously, the composition of the present invention for topical vaginal use consists of:
a high bacterial population, for a fast colonization of the overall ecosystem,
strains of bacteria in a good physiological status, for a prompt multiplication,
a proper stability, under the storage conditions of the final product, so that to ensure the efficacy until the end of the shelf-life.

The composition of the present invention is effectively applicable in the treatment of recurrent or relapsing vaginosis.

Cytokines with Immunoregulatory Action

This study assessed the induction of cytokines IL-4 and IL-10, which represent the main cytokines with immunoregulatory action. As it is shown in figure A, the tested strains of bacteria Lactobacillus plantarum LMG P-21021-LP01 and Lactobacillus plantarum LMG P-21020-LP02 are able to induce a statistically significant growth relative to the basal conditions in both the cytokines.

Figure A: Cytokine profile. Mean±S.E.M. of 10 independent experiments. The statistical meaning is calculated by using the Student's t-test. When calculated relative to the basal conditions (non-stimulated PBMC), the values p<0.05 are considered statistically significant. The IL-10 production was assessed in the culture supernatant after one day from the stimulation. The IL-4 production was assessed in the culture supernatant after five days of stimulation. Similar results were obtained with the strain Lactobacillus plantarum LMG P-21020-LP02.

FIG. 1 relates to the quantification of E. coli (ATCC 8739, ATCC 10536, ATCC 35218, ATCC 25922) inhibition by the strain Lactobacillus plantarum LMG P-21021-LP01.

Figure B relates to the quantification of E. coli (ATCC 8739, ATCC 10536, ATCC 35218, ATCC 25922) inhibition by the strain Lactobacillus plantarum LMG P-21020-LP02.

It is an object of the present invention a composition comprising or, alternatively, consisting of:
a strain of bacterium Lactobacillus plantarum LMG P-21021 (LP01), and/or
a strain of bacterium Lactobacillus fermentum DSM 26955 (LF15), and
a tara gum, and
an arabinogalactan fiber, and
a fructo-oligosaccharide fiber, for use in the preventive and/or curative treatment of bacterial vaginosis, vaginitis and vaginal infections related with coliform pathogens and/or Gardnerella; and for use against recurrent or relapsing vaginosis.

The strain of bacterium Lactobacillus plantarum LMG P-21021 (LP01) and the strain of bacterium Lactobacillus fermentum DSM 26955 (LF15) are each present at a concentration comprised from 1×10⁷ to 1×10¹⁰, preferably from 1×10⁸ to 1×10⁹ CFU/g (and in any case not lesser than 400×10⁶ of viable cells per strain, per tablet or ovule). The two strains of bacteria of Lactobacillus exist in the composition in a weight ratio comprised from 1:3 to 3:1, preferably from 1:2 to 2:1, for example 1:1.

The tara gum is in an amount comprised from 1 to 10% by weight, relative to the weight of the composition, preferably from 3 to 8%, for example 5%.

The arabinogalactan fiber is in an amount comprised from 10 to 45% by weight, relative to the weight of the composition, preferably from 25 to 40%, for example 35%.

The fructo-oligosaccharide fiber is in an amount comprised from 10 to 40% by weight, relative to the weight of the composition, preferably from 15 to 35%, for example 25%.

The weight ratio between arabinogalactan fiber and fructo-oligosaccharide fiber is comprised from 1:2 to 2:1, preferably from 1:1.5 to 1.5:1.

For example, one gram of composition may contain the two strains of bacteria of Lactobacillus at a concentration of 4×10⁶ CFU/g each (weight 370 mg), 50 mg of tara gum, arabinogalactan 340 mg and fructo-oligosaccharide 240 mg; said composition can be in the form of slow-release vaginal tablet (composition).

The Applicant tested both the two strains of bacterium Lactobacillus in vitro and the composition in vivo (study).

i) In vitro said strains of bacterium of Lactobacillus were tested against the strain of G. vaginalis ATCC 10231 grown in BHI (brain-heart infusion broth) containing 2% (w/w) of gelatin, 0.5% of yeast extract, 0.1% of starch and 0.1% of glucose. Each strain was cultured in a culture MRS medium. All the dishes were incubated at 37° C. over 48 hours under anaerobic conditions (GasPak System).

The neutralized supernatants of each strain were added at different percentages to fresh BHI and inoculated with G. vaginalis ATCC 10231. The growth of the strain G. vaginalis alone and in the presence of different concentrations of neutralized supernatant from 0.5% to 4% was quantified by means of optical density at 600 nm (OD₆₀₀) after 24 hours and 48 hours of incubation at 37° C. under microaerophilic conditions.

ii) In vivo said composition was tested in 35 patients divided in: Group A, 24 subjects and Group B (placebo) 11 subjects. The placebo consisted of vaginal tablets of equal weight containing exclusively microcrystalline cellulose and anhydrous calcium hydrogen phosphate (inert ingredients). The vaginal tablets of the composition and placebo were administered to the subjects of Group A and Group B (placebo) once per day for 7 consecutive nights before going to bed. Next, one tablet every 3 days per 3 weeks. The complete treatment lasted 4 weeks for both the groups.

This study was performed in order to at first assess the ability of the selected lactobacilli to antagonize in vitro Gardnerella vaginalis so as to be combined with a strain capable to inhibit Escherichia coli, thus exerting a possible potential use even in aerobic vaginitis (AV). The second step of the study was a human pilot test in women with BV by using a combination of the most promising and active bacteria.

Methods. For this purpose, neutralized supernatants of each lactobacillus were assessed at percentages ranging from 0.5% to 4% for their capability to hindering the growth of G. vaginalis ATCC 10231.

The bacterium capable to exert the greatest inhibition was next tested along with L. plantarum LP01 in a placebo-controlled pilot test, with human interference, which involved 34 female subjects (age between 18 and 50, mean 34.7±8.9, no menopausal women) diagnosed with BV. The two microorganisms L. fermentum LF15 (DSM 26955) and L. plantarum LP01 (LMG P-21021) were administered in the vagina by means of slow-release vaginal tablets also comprising 50 mg of tara gum. The amount of each strain was 400 millions of viable cells per dose. The women were instructed to apply a vaginal tablet once per day for 7 consecutive nights, next a tablet every 3 nights for a further application of 3 weeks (acute stage) and, finally, a tablet per week in order to maintain a long-term vaginal colonization against possible relapses. A clinical examination was performed and the Nugent score quantified for each patient at the beginning (d0), after 28 days (d28) and at the end of the second month for preventing relapses (d56). A statistical comparison between d28, or d56, and d0, as well as between d56 and d28 was performed in order to quantify the efficacy against possible relapses.

Results. L. fermentum LF15 showed the maximum inhibitory activity in vitro against G. vaginalis ATCC 10231 after 24 and 48 hours.

In the human test, the two selected lactobacilli, namely L. fermentum LF15 and L. plantarum LP01, significantly reduced the Nugent score below the threshold of 7 after 28 days in 22 of 24 patients in the Group with active agent (91.7%, p<0.001). Eight women (33.3%) recorded a Nugent score between 4 and 6, evidence of an intermediate situation, whereas the remaining fourteen (58.3%) showed an assessment less to 4, thus suggesting the restoration of a physiological vaginal microbiota. At the end of the second month, only 4 women recorded a Nugent score greater than 7 and which can be defined as BV (16.7%, p=0.065 relative to d28). In the placebo no significant differences were recorded at any time.

The present study suggests the ability of the two strains L. fermentum LF15 and L. plantarum LP01 to effectively hinder acute infections by Gardnerella and to significantly ameliorate the relative annoying symptoms in a very high percentage of women.

This result can be also ascribed to the presence of tara gum, capable to form a mechanical barrier against Gardnerella over the surface of the vaginal mucosa as primary mechanism.

Additionally, a long-term physiological protection is established due to the supplementation of the two lactobacilli in the vaginal microbiota and to their adhesion to the mucosal epithelial cells.

In light of the additional in vitro inhibitory activity against E. coli, their use in aerobic vaginitis (AV) is very useful. The present study shows the ability of L. fermentum LF15 (DSM 26955) and L. plantarum LP01 (LMG P-21021) to effectively hinder acute infections by Gardnerella and to significantly ameliorate the annoying symptoms reported by women with BV. This evidence is ascribed to the presence of tara gum, an ingredient capable to create a mechanical barrier against the adhesion of Gardnerella and other possible detrimental microbes to the surface of the vaginal mucosa. The overall adaptation and tolerability were very good, since only one exclusion in the placebo was recorded due to the deviation from the protocol and no adverse events occurred.

A long-term physiological protection due to the supplementation of the two lactobacilli in the vaginal microbiota and their adhesion to the mucosal epithelial cells is also shown, since a very low percentage of relapses was recorded in the group with active agent during the second month of the protocol. In this regards, the use of arabinogalactan and fructo-oligosaccharides (FOS), two fibers capable to improve the vaginal colonization by the two lactobacilli, has a role in the long-term barrier against reinfections recorded in the second month of the study protocol.

Although the group with placebo included a lesser number of subjects relative to the group with active agent, the placebo effect was very low since no statistically significant differences were recorded neither after 28 days (p=0.619) nor at the end of the protocol (p=0.823), thus suggesting that the improvement recorded in the subjects receiving the two lactobacilli is specific and attributable to the global effect of the tested composition.

It is also interesting to underline the fact that an in vitro function exerted by a selected Lactobacillus, such as the inhibition of the Gardnerella growth by means of specific action mechanisms, was advantageously confirmed during this pilot test in female subjects diagnosed with By. This suggests that the in vitro antagonism against G. vaginalis can prove to be a criterion for effectively selecting lactobacilli with a protective action in the event of BV, as regards both an acute treatment and the long-term prevention of relapses. In the in vitro step of the study, the selection of a proper growth medium suitable for the Gardnerella inhibition test was another key point since presumably certain components existing in MRS, the medium commonly used for growing lactobacilli before the inhibition assessment, can be able to promote the Gardnerella growing. In fact, the growth of Gardnerella in BHI alone was relatively poor, particularly after 48 hours.

In light of the in vitro inhibitory activity against E. coli primarily exerted by L. plantarum LP01 (29), the potential use of its combination with L. fermentum LF15 in aerobic vaginitis (AV), can prove to be highly useful and interesting. In fact, AV is mainly related to Escherichia coli, but also Streptococcus agalactiae and Staphylococcus aureus are involved. The standard AV treatment consists of oral or vaginal antibiotics, although it is unable to automatically restore a normal flora characterized by a high concentration of lactobacilli, thus easing relapses and reinfections. In conclusion, the study also represents an effective approach for preventing possible relapses caused by Gardnerella vaginalis (bacterial vaginosis) or Escherichia coli (aerobic vaginitis).

1. A method to prevent and/or treat a subject, the method comprising:
administering to the subject a composition comprising a mixture of bacteria, which comprises:
at least a strain of bacterium selected from Lactobacillus plantarum LMG P-21021 (LP01) and Lactobacillus plantarum LMG P-21020 (LP02); and
at least a strain of bacterium selected from Lactobacillus fermentum DSM 26955 (LF15) and Lactobacillus fermentum DSM 26956 (LF16),
the mixture of bacteria in an amount effective for preventive and/or curative treatment of bacterial vaginosis, vaginitis and vaginal infections related with coliform pathogens and/or Gardnerella.

2. The method according to claim 1, wherein the vaginitis is recurrent or relapsing vaginosis.

3. The method according to claim 1, wherein said mixture of bacteria comprises the strain of bacterium Lactobacillus plantarum LMG P-21021 (LP01), combined with at least a strain of bacterium selected from Lactobacillus fermentum DSM 26955 (LF15) and Lactobacillus fermentum DSM 26956 (LF16).

4. The method according to claim 3, wherein said mixture of bacteria comprises the strain of bacterium Lactobacillus plantarum LMG P-21021 (LP01) and the strain of bacterium Lactobacillus fermentum DSM 26955 (LF15).

5. The method according to claim 1, wherein said mixture of bacteria comprises the strain of bacterium Lactobacillus plantarum LMG P-21020 (LP02), combined with at least a strain of bacterium selected from Lactobacillus fermentum DSM 26955 (LF15) and Lactobacillus fermentum DSM 26956 (LF16).

6. The method according to claim 5, wherein said mixture of bacteria comprises Lactobacillus plantarum LMG P-21020 (LP02) and Lactobacillus fermentum DSM 26956 (LF16).

7. The method according to claim 1, wherein said composition further comprises at least a gelling substance selected from a tannate or a gelatin tannate, an alginate, a xyloglucan or xylogel, a guar gum, a tara gum, an acacia, carob, oat, bamboo fiber, citrus fruit fibers and glucomannans;

8. The method according to claim 1, wherein said composition further comprises at least a fiber with prebiotic activity selected from inulin, fructo-oligosaccharides (FOS), galacto and trans-galacto-oligosaccharides (GOS and TOS), gluco-oligosaccharides (GOSa), xylo-oligosaccharides, (XOS), chitosan-oligosaccharides (COS), soy-oligosaccharides (SOS), isomalto-oligosaccharides (IMOS), resistant starch, pectins, psyllium, arabino-galactans, gluco-mannans and galacto-mannans.

9. The method according to claim 1, wherein said composition comprises a mixture comprising:
a strain of bacterium Lactobacillus plantarum LMG P-21021 (LP01), and/or
a strain of bacterium Lactobacillus fermentum DSM 26955 (LF15);
a tara gum;
an arabinogalactan fiber; and
a fructo-oligosaccharide fiber.

10. The method according to claim 9, wherein the strain of bacterium Lactobacillus plantarum LMG P-21021 (LP01) and the strain of bacterium Lactobacillus fermentum DSM 26955 (LF15) are each present in the composition in a concentration comprised from 1×10⁷ to 1×10¹⁰ CFU/g of composition.

11. The method according to claim 9, wherein said two strains of bacterium are present in the composition in a weight ratio comprised from 1:3 to 3:1.

12. The method according to claim 9, wherein the tara gum is present in the composition in an amount comprised from 1 to 10% by weight, relative to the weight of the composition.

13. The method according to claim 9, wherein said arabinogalactan fiber is present in the composition in an amount comprised from 10 to 45% by weight, relative to the weight of the composition, and said fructo-oligosaccharide fiber is present in an amount comprised from 10 to 40% by weight, relative to the weight of the composition.

14. The method according to claim 9, wherein the weight ratio between arabinogalactan fiber and fructo-oligosaccharide fiber is comprised from 1:2 to 2:1.

15. A method to prevent and/or treat bacterial vaginosis, vaginitis and vaginal infections related with coliform pathogens and/or Gardnerella in a subject, the method comprising:
administering to the subject an effective amount of a strain of bacterium belonging to the species Lactobacillus fermentum deposited by Probiotical S.p.A. Company on 3 Jan. 2013 at the DSMZ center and having deposit number DSM 26955 (LF15) and a strain of bacterium belonging to the species Lactobacillus fermentum deposited by Probiotical S.p.A. Company on 3 Jan. 2013 at the DSMZ center and having deposit number DSM 26956 (LF16).

16. The method according to claim 7, wherein the gelling substance has a marked muco-adhesive property.

17. The method according to claim 9, wherein the strain of bacterium Lactobacillus plantarum LMG P-21021 (LP01) and the strain of bacterium Lactobacillus fermentum DSM 26955 (LF15) are each present in the composition in a concentration from 1×10⁸ to 1×10⁹ CFU/g of composition.

18. The method according to claim 9, wherein said two strains of bacterium are present in the composition in a weight ratio from 1:2 to 2:1.

19. The method according to claim 9, wherein the tara gum is present in an amount comprised from 3 to 8% by weight, relative to the weight of the composition.

20. The method according to claim 9, wherein said arabinogalactan fiber is present in the composition in an amount comprised from 25 to 40% by weight, relative to the weight of the composition, and said fructo-oligosaccharide fiber is present in an amount comprised from 15 to 35% by weight, relative to the weight of the composition.
